(19) United States
(12) Patent Application Publication
(10) Pub. No.: US 2014/0242611 A1
(43) Pub. Date: Aug. 28, 2014

(54) METHOD FOR DETECTING AND/OR QUANTIFYING AN ANALYTE AT THE SURFACE OF A CELL

(71) Applicants: Hervé Bazin, Villeneuve Les Avignon (FR); Gérard Mathis, Bagnols Sur Ceze (FR)

(72) Inventors: Hervé Bazin, Villeneuve Les Avignon (FR); Gérard Mathis, Bagnols Sur Ceze (FR)

(73) Assignee: CISBIO BIOASSAYS, Codolet (FR)

(21) Appl. No.: 14/131,042

(22) PCT Filed: Jul. 4, 2012
(86) PCT No.: PCT/FR2012/051556

§ 371 Date: Apr. 10, 2014

(30) Foreign Application Priority Data
Jul. 6, 2011 (FR) 1156103

(51) Int. Cl. G01N 33/58

(52) U.S. Cl. CPC G01N 33/582; G01N 2458/00
USPC 435/7.23; 435/7.21

(57) ABSTRACT

The invention relates to a method for quantifying a protein of interest expressed at the surface of a cell or else present in a tissue sample, said method comprising the use of two ligands capable of binding specifically to a domain of said protein.

The invention relates to an improved method for detecting and/or quantifying protein in a sample, in particular a tissue sample.

### STATE OF THE ART

Immunohistochemistry (IHC) is the name given to a method for locating proteins in cells on a tissue section, by detecting antigens by means of antibodies. Immunohistochemistry takes advantage of the fact that an antibody binds specifically to antigens in biological tissues. Antibodies may be of polyclonal or monoclonal origin, monoclonal antibodies being essentially more specific.

An antibody-antigen pair can be visualized in several ways. In most cases, an antibody is conjugated to an enzyme (alkaline phosphatase, horseradish peroxidase in particular) which generates products that are highly colored in the presence of a chromogenic substrate (for instance DAB for horseradish peroxidase) or else a fluorophore (FITC, TRITC, AMCA etc.). Densitometric analysis of the signal obtained with chromogenic or fluorescence methods can provide semi-quantitative or quantitative data, respectively, and makes it possible to correlate the level of the signal measured with the level of expression of proteins or localization. It allows semi-quantitative detection based on observation under a microscope by an anatomopathologist who classifies the section as “negative” or “positive” (1+, 2+ or 3+).

Immunohistochemistry is widely used for the diagnosis and/or follow-up of cancers by detection of abnormal cells such as those found in cancerous tumors. Specific markers are thus today known for various cancers, such as carcinoembryonic antigen (CEA), used in the case of colon cancer, CD15 and CD30, used for Hodgkin's disease, alpha-fetoprotein, used in the case of hepatocellular carcinomas, CD117, a marker of gastrointestinal Stromal tumors, and Ki-67, one of the indicators of tumor proliferation. These molecular markers are characteristic of certain cellular events such as proliferation or cell death (apoptosis). Immunohistochemistry is also widely used in fundamental research for understanding the distribution and localization of biomarkers and of proteins expressed in the various parts of a biological tissue.

Two approaches are used in immunohistochemistry: a direct method and an indirect method. The direct method comprises only one coloration step and is based on the use of a labeled antibody which reacts directly with the antigen in the tissue sections. Although this technique is simple and rapid, it has a low sensitivity because of the absence of amplification of the signal. The indirect method consists in using an unlabeled primary antibody, specific for the antigen of interest, and a labeled secondary antibody which recognizes the IgGs of the animal species in which the primary antibody was prepared. This method is more sensitive than direct detection strategies because of the amplification of the signal due to the binding of several secondary antibodies to each primary antibody.

Conventional immunohistochemistry techniques require several steps before the final coloration of the tissue antigen, and many potential problems can affect the result of the procedure. The problems most frequently encountered are a background noise which is too high, insufficient labeling of the antigen and autofluorescence problems. To reduce the background noise related to nonspecific binding of the primary or secondary antibody to proteins present in the sample, the latter are generally incubated with a buffer which blocks the reactive sites to which the primary or secondary antibody may bind. The blocking buffers most commonly used are: normal serum, nonfat powdered milk, bovine serum albumin (BSA) or gelatin, and other buffers with specific formulations are also commercially available.

There is a need for a technique for analyzing biological samples, in particular of tissues, which does not have the drawbacks of the conventional techniques in terms of background noise in particular.

The invention makes it possible to solve these problems; it in particular drastically reduces the background noise observed when conventional immunohistochemistry approaches are used, namely when a single antibody is used for detecting the protein of interest.

### DESCRIPTION

An object of the invention is a method advantageously using a pair of FRET partners for quantifying proteins present at the surface of a cell or in a tissue sample. An object of the invention is also a kit of reagents for implementing this method, and the application of this method to the HER2 protein in the context of a theranostic method for determining whether patients suffering from breast cancer are eligible for treatment with an anti-HER2 antibody.

The term “pair of FRET partners” is intended to mean a pair consisting of an energy donor fluorescent compound (hereinafter “donor fluorescent compound”) and an energy acceptor compound (hereinafter “acceptor compound”); when they are in proximity to one another and when they are excited at the excitation wavelength of the donor fluorescent compound, these compounds emit a FRET (“Forster Resonance Energy Transfer”) signal.

The term “FRET signal” is intended to mean any measurable signal representative of a FRET between a donor fluorescent compound and an acceptor compound. A FRET signal can thus be a variation in the intensity or the life time of the luminescence of the donor fluorescent compound or of the acceptor compound when the latter is fluorescent. When the FRET signal is measured in resolved time (which is generally the case when rare earth chelates or cryptates are used), the term TR-FRET (acronym of time-resolve FRET) is used.

A first aspect of the invention relates to a method for quantifying a protein of interest expressed at the surface of a cell or else present in a tissue sample, comprising the following steps:

(i) bringing cells or a tissue sample expressing said protein into contact with a first and a second ligand, each of these ligands being capable of binding specifically to a domain of said protein, and these ligands being respectively labeled with a donor compound and an acceptor compound, both forming a pair of FRET partners;
(ii) washing the cells or the tissue sample;
(iii) measuring the FRET signal emitted by the measuring medium.

The term “tissue sample” is preferably intended to mean a solid tissue sample taken from a patient, and preferably a tumor tissue extract.

The term “method for quantifying” is intended to mean a method for relative quantification, i.e. the signal measured will be different from one sample to another, depending on the amount of protein of interest present in the sample.

This technique differs from the conventional immunohistochemistry methods in so far as it uses two ligands (in particular two antibodies) specific for the protein of interest, and not just one, as in the prior art methods. It also comprises a washing step which is not generally carried out when the FRET technique is used, since this approach allows measurements in a homogeneous medium. This method, combined with the fact that the FRET signal will be emitted only when these ligands (or antibodies) are in proximity to one another, results in a signal/noise ratio which is much better than that observed with the conventional protocols, namely those using a single ligand or antibody.

When the method is carried out on a tissue sample, it requires steps for preparing this sample in the form of sections 20 to 50 μm thick. These preparation steps are those which are conventionally carried out in the field of those skilled in the art, namely immunohistochemistry. They can consist in particular in fixing the sample via a treatment with formaldehyde, and embedding said sample in paraffin, in particular in the form of blocks which can subsequently be cut on a microtome (preferably with a thickness of approximately 20 to 50 μm). Treatment of these sections with xylene in order to remove the paraffin, and rinsing of said sections with ethanol and then with water are also techniques known to those skilled in the art, as is regeneration of the epitopes by means of the HIER (acronym of “heat induced epitope recovery”) technique.

Alternatively, the method according to the invention can also be carried out on cryosections, preferably 20 to 50 μm thick, also prepared according to conventional techniques.

When the method according to the invention is carried out on a tissue sample, it preferably comprises a step aimed at homogenizing this sample in the form of a cell lysate, before or after the introduction of the fluorescent compounds into the measuring medium. This step is preferentially carried out after the introduction of the fluorescent compounds into the measuring medium (first ligand, second ligand and optionally labeling agent), and before the measurement of the FRET signal. Such a treatment may be mechanical, and may be chosen from: the application of ultrasound (sonication), freezing/thawing cycles, the use of mechanical grinders, optionally together with the use of a hypotonic lysis buffer or of a lysis buffer containing detergents, such as the RIPA buffer.

Moreover, when the method according to the invention is carried out on a tissue sample, it has been determined that the final concentrations of first and second ligand in the measuring medium are, optimally, greater than 10 nM, preferably between 10 and 150 nM or between 20 and 80 nM, and preferably between 30 and 60 nM. The term “final concentration” is intended to mean the concentration of these compounds in the measuring medium once all the reagents have been introduced into this medium. These concentration ranges are notably higher than the concentrations normally used in assays of TR-FRET type, in which the final concentrations of fluorescent ligands are of the order of one nanomolar, i.e. less than 10 nM.

The method may also be carried out on adherent cells, or even on cells in suspension. In the latter case, nevertheless, at least one centrifugation step will be required for carrying out the washing step.

It may be advantageous to normalize the FRET signal with respect to the amount of biological material (cells or tissue) present in the measuring medium. The method according to the invention thus comprises, in one particular embodiment, incubating the cells or the tissue sample with an agent for fluorescent labeling of DNA (for example, Hoechst 33342), prior to the washing step, and the FRET signal will be normalized with respect to the signal corresponding to the luminescence of this labeling agent.

In one preferred embodiment, the first and the second ligand are antibodies which recognize the protein of interest. The term “antibody” should here be taken in the broad sense and comprises any protein of the immunoglobulin family or else comprising an immunoglobulin domain, and also a site for specific binding to the protein of interest. The antibodies may therefore be Fab or Fab′ fragments, single-chain antibodies, or variable domains of immunoglobulin heavy or light chains.

Those skilled in the art are able to produce antibodies specific for the protein of which they desire to determine the expression using conventional techniques. Many antibodies are also commercially available. Those skilled in the art will take care to select antibodies which recognize different epitopes on the protein of interest, so as to enable simultaneous binding of said antibodies to this protein.

Other ligands may be used in place of the antibodies. Thus, in one particular embodiment of the invention, the first ligand is a known ligand (such as an agonist or antagonist) of the protein of interest and is not an antibody, and the second ligand is an antibody. Here again, it is preferable for the binding site for the first ligand to be different from the binding site for the antibody.

If the protein of interest is a G-protein-coupled receptor, those skilled in the art will be able to refer to the database published by Okuno et al. (GLIDA: GPCR ligand database for chemical genomics drug discovery database and tools update. Nucl. Acids Res., 36(suppl_{—}1), D907-912) for finding ligands that can be used in the method according to the invention.

The protein of interest may be any protein expressed by the cells present in the measuring medium. The method is particularly suitable for studying membrane proteins, such as ion channels, receptors, in particular G-protein-coupled receptors (GPCRs), receptor tyrosine kinases (RTKs), such as, for example: EGFR (HER1), HER2, HER3, HER4.

The method is particularly useful for determining the expression of the HER2 protein in tissue samples, in particular tumor samples, for determining whether the patients concerned are eligible for anti-HER2 type treatment, in particular with an antibody which binds to the HER2 protein, such as trastuzumab (Herceptin™). For this, the value obtained representing the expression of HER2 is compared with a reference threshold value above which the patients are directed toward an anti-HER2 type therapy. In a second aspect, the invention therefore relates to a method for selecting patients suffering from cancer who are eligible for therapeutic treatment with an antibody which binds to the HER2 protein, which method comprises the implementation of the method of quantification according to the invention in which the protein is HER2.

Entirely surprisingly, the method according to the invention has made it possible to detect the HER2 protein unequivocibly in “HercepTest™ negative” patients, namely in patients whose results in this test made them ineligible for treatment with the trastuzumab antibody (patients classified 0, 1+ or 2+). Thus, in one embodiment particularly useful from a clinical point of view, the method according to the invention is carried out on a tissue sample from a patient for whom the results of immunohistochemical analysis of the expression of the HER2 protein are negative (0, 1+ and 2+). This method is particularly invaluable for patients having undergone hormonal chemotherapy, in particular when the latter has not brought about an improvement in their condition.

In one preferred embodiment, the protein of interest is different from a receptor expressed constitutively and exclusively in homodimer form.

When the protein of interest is the EGFR (HER1) receptor, one of the ligands labeled with the FRET partners may be an anti-EGFR antibody and the other ligand may be EGF or an anti-EGFR antibody (anti-EGFR antibodies being commercially available).

When the protein structure of interest is the HER2 protein, the first and second ligands are preferably antibodies specific for the HER2 protein, in particular antibodies of which the epitopes are located in the extracellular domain of this receptor. As indicated above, it is preferable for these epitopes to be different.

In a third aspect, the invention relates to a kit of reagents for implementing the method according to the invention. This kit of reagents contains a first and a second ligand, each of these ligands being capable of binding specifically to a domain of a membrane protein of interest, and these ligands being respectively labeled with a donor compound and an acceptor compound, both forming a pair of FRET partners. The technical characteristics of these ligands are those described above.

A kit of reagents for quantifying the HER2 protein according to the invention is of considerable therapeutic benefit to patients judged to be ineligible for anti-HER2 type treatment using the conventional IHC test (HercepTest™). Such a kit, in which the ligands are antibodies specific for the HER2 protein, is therefore particularly preferred.

Labeling of the Antibodies with Energy Donor or Acceptor Compounds

The labeling of a ligand or of an antibody with a fluorescent donor or acceptor compound is carried out by conventional conjugation techniques making use of reactive groups. The fluorescent donor or acceptor compounds are generally sold in “functionalized” form, i.e. they bear a reactive group capable of reacting with a functional group present on the compound to be labeled, in this case the ligand.

Typically, the reactive group present on the donor or acceptor fluorescent compound is an electrophilic or nucleophilic group which can form a covalent bond when it is placed in the presence of an appropriate nucleophilic or electrophilic group, respectively. By way of examples, the pairs of electrophilic/nucleophilic groups and the type of covalent bond formed when they are placed in the presence of one another are listed below:

| Electrophilic | Nucleophilic | |
|---|---|---|
| group | group | Type of bond |
| acrylamides | thiols | thioethers |
| acyl halides | amines/anilines | carboxamides |
| aldehydes | amines/anilines | imines |
| aldehydes or | hydrazines | hydrazones |
| ketones | | |
| aldehydes or | hydroxylamines | oximes |
| ketones | | |
| alkyl sulfonates | thiols | thioethers |
| anhydrides | amines/anilines | carboxamides |
| aryl halides | thiols | thioethers |
| aryl halides | amines | aryl amines |
| aziridines | thiols | thioethers |
| carbodiimides | carboxylic acids | N-acylureas or |
| | | anhydrides |
| activated esters* | amines/anilines | carboxamides |
| haloacetamides | thiols | thioethers |
| halotriazines | amines/anilines | aminotriazines |
| imido esters | amines/anilines | amidines |
| isocyanates | amines/anilines | ureas |
| isothiocyanates | amines/anilines | thioureas |
| maleimides | thiols | thioethers |
| sulfonate esters | amines/anilines | alkyl amines |
| sulfonyl halides | amines/anilines | sulfonamides |

*The term “activated ester” is intended to mean groups of formula COY, where Y is:
a leaving group, chosen from succinimidyloxy (—OC₄H₄NO₂) and sulfosuccinimidyloxy (—OC₄H₃NO₂—SO₃H) groups;
an aryloxy group which is unsubstituted or substituted with at least one electrophilic substituent, such as nitro, fluoro, chloro, cyano or trifluoromethyl groups, thus forming an activated aryl ester;
a carboxylic acid activated by a carbodiimide group, forming an anhydride —OCORa or —OCNRaNHRb, in which Ra and Rb are identical or different and are chosen from C₁-C₆ alkyl, C₁-C₆ perfluoroalkyl, C₁-C₆ alkoxy, and cyclohexyl groups;
3-dimethylaminopropyl or N-morpholinoethyl.

The commercially available donor and acceptor fluorescent compounds generally comprise a maleimide function or an activated ester, most commonly activated with an NHS (N-hydroxysuccinimidyl) group, which react with thiol and amine groups, respectively, and can therefore be used for the labeling of antibodies. The labeled antibodies are characterized by the final molar ratio (FMR) which represents the average number of label molecules grafted to the ligand.

When the ligand is protein in nature, it may be advantageous to use one of the functional groups naturally present in proteins: the amino-terminal group, the carboxylate terminal group, the carboxylate groups of aspartic acid and glutamic acid, the amine groups of lysines, the guanidine groups of arginines, the thiol groups of cysteines, the phenol groups of tyrosines, the indole rings of tryptophans, the thioether groups of methionines, the imidazole groups of histidines.

If the ligand does not comprise a functional group in the natural state, such groups can be introduced. Methods for introducing functional groups are in particular described in C. Kessler, Nonisotopic probing, Blotting and Sequencing, 2nd edition, L. J. Kricka (1995), Ed. Academic press Ltd., London, p. 66-72.

Another approach for labeling a ligand with a fluorescent compound consists in introducing a reactive group into the ligand, for example an NHS group or a maleimide group, and placing it in the presence of a fluorophore bearing a functional group that will react with the reactive group so as to form a covalent bond.

It is important to verify that the labeled ligand retains a sufficient affinity for its receptor; this can be controlled simply by means of conventional binding experiments, making it possible to calculate the affinity constant of the labeled ligand for the receptor.

### Pairs of FRET Partners

The pairs of FRET partners preferably consist of an energy donor fluorescent compound and an energy acceptor fluorescent compound.

FRET is defined as a transfer of nonradiative energy resulting from a dipole-dipole interaction between an energy donor and an energy acceptor. This physical phenomenon requires energy compatibility between these molecules. This means that the emission spectrum of the donor must at least partially overlap the absorption spectrum of the acceptor. In accordance with Forster's theory, FRET is a process which depends on the distance separating the two donor and acceptor molecules: when these molecules are in proximity to one another, a FRET signal will be emitted.

The selection of the donor/acceptor fluorophore pair for obtaining a FRET signal is within the reach of those skilled in the art. Donor-acceptor pairs that can be used for studying FRET phenomena are in particular described in the textbook by Joseph R. Lakowicz (Principles of fluorescence spectroscopy, 2nd edition, Kluwer academic/plenum publishers, NY (1999)), to which those skilled in the art will be able to refer.

Long-life (>0.1 ms, preferably between 0.5 and 6 ms) energy donor fluorescent compounds, in particular rare earth chelates or cryptates, are advantageous since they make it possible to perform time resolved measurements, i.e. to measure TR-FRET (Time Resolved FRET) signals while dispensing with the phenomenon of autofluorescence emitted by the measuring medium. For this reason, they are generally preferred for carrying out the method according to the invention.

Dysprosium (Dy3+), samarium (Sm3+), neodymium (Nd3+), ytterbium (Yb3+) or else erbium (Er3+) complexes are rare earth complexes which are equally suitable for the purposes of the invention, but europium (Eu3+) chelates and cryptates and terbium (Tb3+) chelates and cryptates are particularly preferred.

A very large number of rare earth complexes have been described, and several are currently sold by the companies PerkinElmer, Invitrogen and Cisbio Bioassays.

Examples of rare earth chelates or cryptates that are suitable for the purposes of the invention are:
Lanthanide cryptates comprising one or more pyridine units. Such rare earth cryptates are described, for example, in patents EP 0 180 492, EP 0 321 353 and EP 0 601 113 and in international application WO 01/96 877. Terbium (Tb3+) and europium (Eu3+) cryptates are particularly suitable for the purposes of the present invention. Lanthanide cryptates are sold by the company Cisbio Bioassays. By way of nonlimiting example, mention may be made of the europium cryptates having the formulae below (which can be coupled to the compound to be labeled via a reactive group, in this case, for example, an NH₂ group):

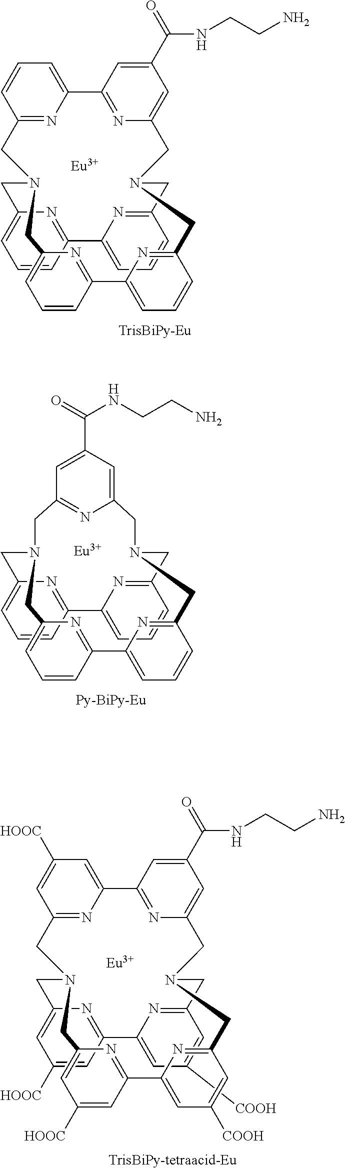

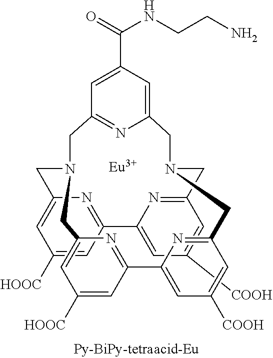

The lanthanide chelates described in particular in patents U.S. Pat. No. 4,761,481, U.S. Pat. No. 5,032,677, U.S. Pat. No. 5,055,578, U.S. Pat. No. 5,106,957, U.S. Pat. No. 5,116,989, U.S. Pat. No. 4,761,481, U.S. Pat. No. 4,801,722, U.S. Pat. No. 4,794,191, U.S. Pat. No. 4,637,988, U.S. Pat. No. 4,670,572, U.S. Pat. No. 4,837,169 and U.S. Pat. No. 4,859,777. Patents EP 0 403 593, U.S. Pat. No. 5,324,825, U.S. Pat. No. 5,202,423 and U.S. Pat. No. 5,316,909 describe chelates composed of a nonadentate ligand such as terpyridine. Lanthanide chelates are sold by the company PerkinElmer.
Lanthanide complexes consisting of a chelating agent, such as tetraazacyclododecane, substituted with a chromophore comprising aromatic rings, such as those described by R. Poole et al., in Biomol. Chem., 2005, 3, 1013-1024 “Synthesis and characterization of highly emissive and kinetically stable lanthanide complexes suitable for usage in cellulo”, can also be used. The complexes described in application WO 2009/10580 can also be used.
The lanthanide cryptates described in patents EP 1 154 991 and EP 1 154 990 can also be used.
The terbium cryptate having the formula below (which can be coupled to a compound to be labeled via a reactive group, in this case, for example, an NH₂ group):

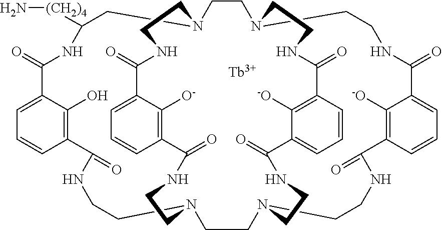

and the synthesis of which is described in international application WO 2008/063721 (compound 6a, page 89).
The terbium cryptate Lumi4-Tb from the company Lumiphore, sold by Cisbio Bioassays.
The quantum dye from the company Research Organics, having the formula below (which can be coupled to the compound to be labeled via a reactive group, in this case NCS):

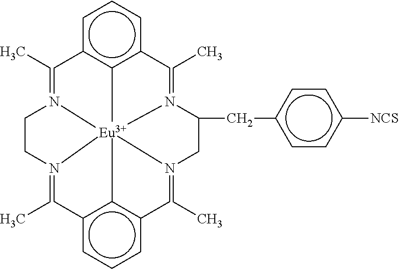

Ruthenium chelates, in particular the complexes consisting of a ruthenium ion and of several bipyridines, such as ruthenium(II) tris(2,2′-bipyridine).
The terbium chelate DTPA-cs 124 Tb, sold by the company Life Technologies, having the formula below (which can be coupled to the compound to be labeled via a reactive group R) and the synthesis of which is described in U.S. Pat. No. 5,622,821.

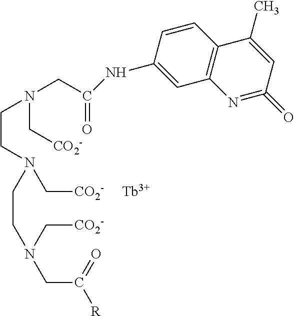

The terbium chelate having the formula below and described by Latva et al (Journal of Luminescence 1997, 75: 149-169):

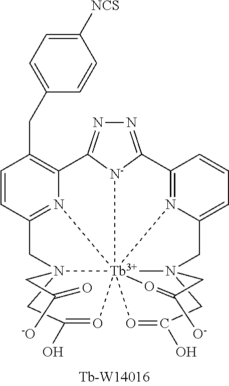

Particularly advantageously, the donor fluorescent compound is chosen from: a europium cryptate; a europium chelate; a terbium chelate; a terbium cryptate; a ruthenium chelate; and a quantum dye; europium chelates and cryptates and terbium chelates and cryptates being particularly preferred.

Dysprosium (Dy3+), samarium (Sm3+), neodymium (Nd3+), ytterbium (Yb3+) or else erbium (Er3+) complexes are also rare earth complexes that are suitable for the purposes of the invention.

The acceptor fluorescent compounds may be chosen from the following group: allophycocyanins, in particular those known under the trade name XL665; luminescent organic molecules, such as rhodamines, cyanines (for instance Cy5), squaraines, coumarins, proflavins, acridines, fluoresceins, boron-dipyrromethene derivatives (sold under the name “Bodipy”), fluorophores known under the name “Atto”, fluorophores known under the name “DY”, compounds known under the name “Alexa”, and nitrobenzoxadiazole. Advantageously, the acceptor fluorescent compounds are chosen from allophycocyanins, rhodamines, cyanines, squaraines, coumarins, proflavins, acridines, fluoresceins, boron-dipyrromethene derivatives and nitrobenzoxadiazole.

The expressions “cyanines” and “rhodamines” should be respectively understood as “cyanine derivatives” and “rhodamine derivatives”. Those skilled in the art know these various fluorophores, which are commercially available.

The “Alexa” compounds are sold by the company Invitrogen; the “Atto” compounds are sold by the company Atto-tec; the “DY” compounds are sold by the company Dyomics; the “Cy” compounds are sold by the company Amersham Biosciences; the other compounds are sold by various suppliers of chemical reagents, such as the companies Sigma, Aldrich or Acros.

The following fluorescent proteins may also be used as acceptor fluorescent compound: cyan fluorescent proteins (AmCyanl, Midori-Ishi Cyan, mTFP1), green fluorescent proteins (EGFP, AcGFP, TurboGFP, Emerald, Azami Green, ZsGreen), yellow fluorescent proteins (EYFP, Topaz, Venus, mCitrine, YPet, PhiYFP, ZsYellowl, mBanana), orange and red fluorescent proteins (Orange kusibari, mOrange, tdtomato, DsRed, DsRed2, DsRed-Express, DsRed-Monomer, mTangerine, AsRed2, mRFP1, JRed, mCherry, mStrawberry, HcRedl, mRaspberry, HcRed-Tandem, mPlim, AQ143), far-red fluorescent proteins (mKate, mKate2, tdKatushka2).

For the purposes of the invention, the cyanine derivatives or the fluorescein derivatives are preferred as acceptor fluorescent compounds.

### EXAMPLES

### Example 1

NIH/3T3 cells (mouse fibroblasts not expressing the human EGFR receptor) were stably transfected with a plasmid containing the sequencing encoding hEGFR, and selected in a medium containing puromycin. The line obtained, expressing the hEGFR receptor, will subsequently be called P1. Similarly, (with the exception of the selection medium which contained hygromycin), a line expressing the human HER2 protein was prepared (hereinafter “H2” line).

The tumor obtained by xenografts of P1 cells on a mouse and fixed in formaldehyde according to a conventionally used protocol was subsequently embedded in paraffin. The resulting blocks were cut on a microtome (thickness ˜20 μm) and the resulting FFPE (“Formaldehyde Fixed Paraffin Embedded”) sections were stored in an eppendorf tube at 4° C. until use.

1.2 ml of xylene substitute were added to a section contained in an eppendorf tube and, after incubation for 5 min, the tube was centrifuged (16 000 RCF) and the supernatant was removed by pipetting. The same process was repeated once, and then 1.2 ml of absolute ethanol were added. After incubation for 3 min, the tube was centrifuged (16 000 RCF) and the supernatant was removed by pipetting. The same process was repeated once, and then 1.2 ml of 95% ethanol were added. After incubation for 3 min, the tube was centrifuged (16 000 RCF) and the supernatant was removed by pipetting. Next, 1.2 ml of 50% ethanol were added, and after incubation for 3 min, the tube was centrifuged (16 000 RCF) and the supernatant was removed by pipetting. 0.5 ml of 10 mM TRIS-HCl+EDTA buffer (pH9) was added and the tube was then closed and heated in a water bath for 20 min. After cooling (10 min), the tube was centrifuged (16 000 RCF) and the supernatant was removed by pipetting.

The section, the EGFR epitopes of which were regenerated by heat treatment (HIER for Heat Induced Epitope Recovery), was resuspended in an incubation buffer consisting of 180 μl of 50 mM HEPES buffer, pH7, containing a cocktail of protease inhibitors, 1% of BSA, and a final concentration of 50 nM of an EGFR-specific antibody Ab15 (Thermo Fisher) labeled with Lumi4® Tb cryptate (Ab15-Lumi4Tb®, Cisbio Bioassays) and of 50 nM of an antibody REGF01, also specific for EGFR (Cisbio Bioassays) and labeled with the acceptor fluorophore d2 (Cisbio Bioassays), which emits at 665 nm (REGF01-d2).

After incubation for 16 h at 20° C., 20 μl of a solution of Hoechst 33342 at 20 μg/ml in buffer were added, and then, after incubation for 15 min, the tube was centrifuged (16 000 RCF) and the supernatant was removed by pipetting. The pellet was resuspended in 300 μl of 50 mM HEPES buffer containing a cocktail of protease inhibitors and 0.1% of BSA, and the tube was centrifuged (16 000 RCF). The cycle of washing/centrifugation in the same buffer was repeated twice, and the supernatants were removed. After sonication (5 s at 20% intensity on a Branson sonicator) enabling homogenization, 100 μl of the lysate obtained were pipetted in the B3 and B4 wells of a black (96-well) microplate.

A “negative control” sample (not expressing the EGFR receptor) was prepared in a similar manner, but from a tumor itself obtained by xenografts of H2 cells on a mouse. 100 μl of the lysate obtained were pipetted into each of the B1 and B2 wells of the black microplate so as to constitute a negative control.

Also deposited in the adjacent wells were buffer alone (BB: buffer blank) and 100 μl of diluted solutions of the mixture of REGF01-d2 and Ab15-Lumi4Tb® antibodies (incubation buffer) obtained by cascade ½ dilution (in HEPES buffer, 0.1% BSA) so as to obtain a labeled-antibody final concentration range of from 0.16 nM to 5 nM (wells A3 to A12). Measurement was then carried out successively in fluorescence mode (“prompt fluorescence” delay=0; measurement time after delay=20 μs) on a Tecan Safire 2 device, while exciting the acceptor (d2) at 640 nm and reading the fluorescence emitted at 665 nm, and then the fluorescence in time resolved mode simultaneously at 620 nm (Lumi4Tb®) and 665 nm (delay=60 μs; measurement time after delay=400 μs) on a Pherastar device (excitation by flash lamp) after excitation at 340 nm.

The measurements in fluorescence mode at 665 nm (F665) of the wells of the range (0.16 nM to 5 nM) make it possible to obtain a calibration line linking the fluorescence corrected for each well by subtraction of the fluorescence of the buffer alone (B665): F665c=F665-B665 (see FIG. 1).

The concentration of antibodies bound to the biological material is obtained by interpolation.

TABLE 1

| | |
|---|---|
| H2 cells | P1 cells |
| (no hEGFR) | (hEGFR) |

| | | | | |
|---|---|---|---|---|
| F665c | 28 070 | 28 080 | 45 370 | 45 200 |
| Ab-d2 | 2.8 | 2.8 | 4.6 | 4.6 |
| (nM) | | | | |

The ratio of the specific signal (binding of REGF01-d2 to EGFR) to the negative-control signal is 4.6/2.8-1.64, which is a relatively low signal/noise ratio.

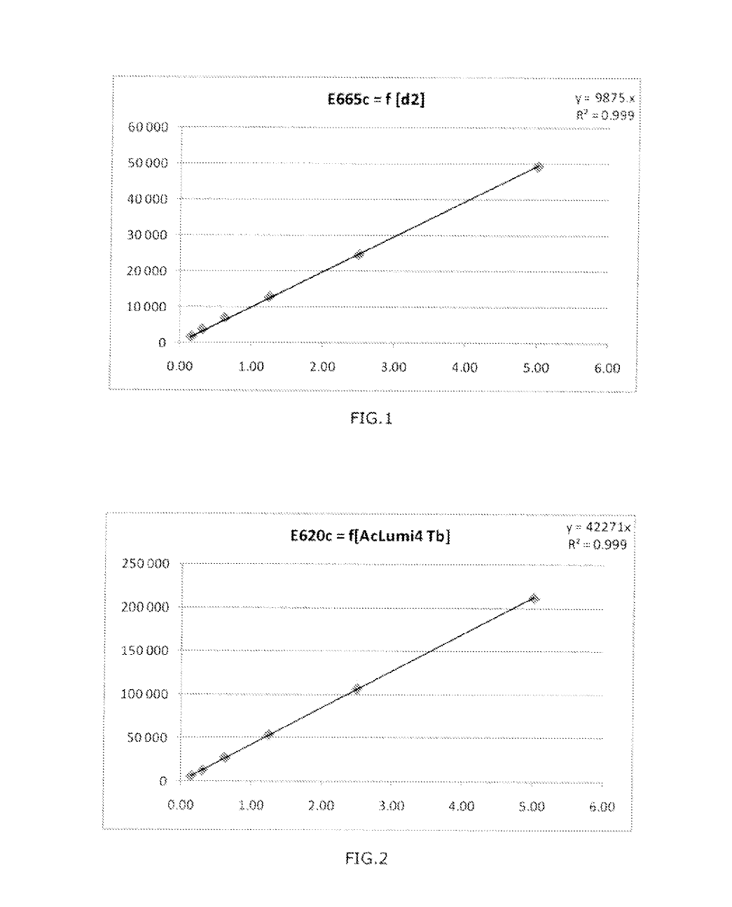

Similarly, the measurements in TRF mode at 620 nm of the wells of the range (0.16 nM to 5 nM) make it possible to obtain a calibration line linking the fluorescence corrected for each well by subtraction of the fluorescence of the buffer alone (B620): E620c=E620-B620 (see FIG. 2).

TABLE 2

| | |
|---|---|
| H2 cells | P1 cells |
| (no hEGFR) | (hEGFR) |

| | | | | |
|---|---|---|---|---|
| E620c | 33 930 | 33 300 | 115 500 | 114 100 |
| AbLumi4Tb (nM) | 0.80 | 0.79 | 2.73 | 2.70 |

The ratio of the specific signal (binding of Ab15-Lumi4Tb® to EGFR) to the negative-control signal is 2.72/0.8=3.4, which is also relatively poor.

The plate is measured in TRF mode simultaneously at 620 nm (luminescence of the Lumi4Tb® donor) and 665 nm (luminescence of the d2 acceptor).

The following values, reported in table 3, are obtained for the calibration range.

TABLE 3

| | Ab Lumi4Tb (nM) | | | | | |
|---|---|---|---|---|---|---|
| | 0.16 | 0.31 | 0.63 | 1.25 | 2.5 | 5 |
| E620 | 6 650 | 13 200 | 27 270 | 53 600 | 106 450 | 210 700 |
| E665 | 609 | 1 150 | 2 360 | 4 630 | 9 260 | 18 250 |

The graph representing E665=f(E620) is plotted, and a straight line is obtained, the slope of which makes it possible to calculate the contribution of the residual emission of the terbium at 665 nm from the signal measured at 620 nm for the H2 and P1 samples, which is called B665 in the following table.

The FRET signal restored at 665 nm is calculated by calculating the difference between the signal at 665 nm (E665c) and the residual emission of the terbium in the 665 nm channel: Δ665=E665c−B665.

In the table below, H460c corresponds to the fluorescence at 460 nm of the Hoechst 33342 compound, measured in conventional fluorescence mode.

TABLE 4

| | |
|---|---|
| H2 cells | P1 cells |
| (no hEGFR) | (hEGFR) |

| | | | | |
|---|---|---|---|---|
| E665 | 4040 | 4060 | 169 700 | 164 200 |
| E665c | 3 875 | 3 900 | 169 560 | 164 060 |
| B665 | 2 920 | 2 860 | 9 940 | 9 8100 |
| Δ665 | 960 | 1 035 | 159 630 | 154 250 |
| H460c | 32 300 | 34 100 | 57 630 | 60 270 |
| Δ665N | 297 | 303 | 27 699 | 25 593 |

| | | |
|---|---|---|
| Mean Δ665N | 300 | 26 646 |

The ratio of the specific signal obtained according to the invention (binding of REGF01-d2 and Ab15-Lumi4Tb0 to EGFR) to the negative-control signal is 26646/300=89, which is excellent.

This example shows that the method according to the invention makes it possible to obtain an excellent signal/noise ratio (factor 89), whereas the use of a single labeled antibody makes it possible to obtain only a factor of 3.42, at best, between the specific signal and the background noise.

### Example 2

Cells of the A431 line (ATCC/CRL-1555 expressing approximately 2×10⁶ EGFR receptors per cell (Shinobu, 1984 Molecular and Cellular Endocrinology 37, 205)) were dispensed into the wells of a microtitration plate (flat-bottomed 96-well black plate) at a density of 25 000 cells per well and incubated over night at 37° C. in DMEM medium so as to obtain a layer of adherent cells.

After two washes with 100 μl of PBS, 50 μl of Krebs buffer were added per well. [Krebs/HEPES buffer mmol/l: NaCl99; KCl4.69; CaCl₂ 1.87; MgSO₄ 1.2; K₂HPO₄ 1.03; NaHCO₃ 25; Na-HEPES 20+glucose 11.1 mM+0.1% BSA, pH 7.4].

No other reagent was added to wells A1 to A4 (buffer blank). 10 μl of 1 μm EGF in Krebs were added to wells A9 to A12 (to saturate the EGF-binding sites), then a mixture of antibody Ab10-d2 (anti-EGFR Ab10 Thermo Fisher Scientific labeled with d2-NHS, Cisbio Bioassays, at an RMF=1.8) and of EGF-Lumi4Tb (Cisbio Bioassays) was added to wells A5 to A12. The volume of each well was made up to 100 μl with Krebs buffer so as to obtain a final concentration in the wells of 5 nM of Ab10-d2 and 5 nM of EGF-Lumi4Tb.

In wells of the same microtitration plate, a standard range was formed by dilution, in Krebs buffer, of a stock solution containing EGF-Lumi4Tb and Ab10-d2 (cascade ½ dilution so as to obtain final Ab concentrations of 1 nM to 0.031 nM). Reading in time resolved mode was carried out on a Pherastar FS device (BMG) using the following parameters:

| | |
|---|---|
| Number of flashes per well | 300 |
| Optical module | HTRF |
| Excitation (nm) | 337 |
| Emission A (nm) | 665 |
| Emission B (nm) | 620 |
| Start of integration [μs]: | 60 |
| Integration time [μs]: | 400 |
| Focusing height [mm]: | 3.9 |
| Multiplier ratio: | 10 000 |
| Light source: | flash lamp |

The E620c emission values obtained (after subtraction of the value of the B620 blank measured on wells A1 and A2 containing only buffer) have been reported in the following table.

TABLE 5

EGF-Lumi 4 Tb (nM)

| | 0.031 | 0.063 | 0.125 | 0.25 | 0.5 | 1 |
|---|---|---|---|---|---|---|
| E620c | 634 | 930 | 1 490 | 3 050 | 5 500 | 10 850 |

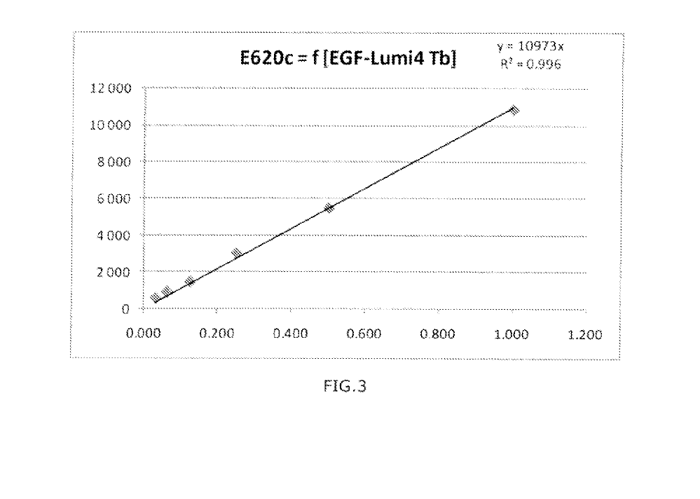

The graph E620c=f (EGF-Lumi4Tb) made it possible to obtain the correspondence between E620c and the concentration in nM of EGF-Lumi4Tb (see FIG. 3).

The plate was incubated for 4 hours at 20° C. (in the dark), before the addition of 10 μl of Hoechst 33342 (solution at 2 mg/ml in DMSO prediluted extemporaneously to 1/100^{th} in Krebs) to wells A3 to A12 and incubation for a further 1 hour at 20° C. All the wells were then washed with four times 100 μl of Krebs buffer and 100 μl of the same buffer added to each well. A time-resolved fluorescence reading was carried out with the same parameters as above.

TABLE 6

| | Well | | | | | | | |
|---|---|---|---|---|---|---|---|---|
| | | | | | 9 | 10 | 11 | 12 |
| | 5 | 6 | 7 | 8 | +EGF (100 nM final | | | |
| | No EGF added | | | | concentration) | | | |
| E620c | 3 870 | 4 710 | 3 390 | 3 280 | 1 180 | 1 340 | 770 | 910 |

| | | |
|---|---|---|
| Mean | 3 810 | 1 050 |
| E620c | | |
| EGF- | 0.348 | 0.096 |
| Lumi4Tb | | |
| (nM) | | |

The wells to which EGF (100 nM final concentration) was added in order to saturate the binding sites of the EGFR receptors expressed by the cells (wells A9 to A12) made it possible to obtain an E620c fluorescence value (mean=1050 AFU) which represents the background noise corresponding to the nonspecific binding of the Lumi4Tb-labelled EGF to the cells. By virtue of the calibration line, it was evaluated that the binding of EGF-Lumi4Tb is of the order of 0.096 nM. The specific binding of EGF-Lumi4Tb to the cells (wells A5 to A8) is of the order of 0.348 nM. The signal/noise ratio when a single ligand of the receptor of interest is used (EGF-Lumi4Tb) is therefore 3.6, which is relatively low.

In order to take into account the variability in the number of cells in the wells (due in particular to the detachment of the cells following the washing), a normalization using the signal of Hoechst 33342 was carried out by dividing the E620c signal by the fluorescence value at 460 nm measured in “conventional” fluorescence mode for the Hoechst/DNA complex in each well (and by multiplying by 100 000 so as to obtain whole numbers).

TABLE 7

| | | | | | | | | |
|---|---|---|---|---|---|---|---|---|
| H460c | 67 380 | 99 330 | 79 590 | 106 300 | 82 990 | 109 800 | 74 400 | 88 000 |
| E620N | 5 750 | 4 740 | 4 250 | 3 090 | 1 420 | 1 220 | 1 040 | 1 040 |

| | | |
|---|---|---|
| Mean | 4 460 | 1 180 |
| E620N | | |

The signal/noise ratio is 3.78 after normalization of the values with respect to the signal of Hoechst 33342, which is also relatively low.

Measurement of the Binding of the Labeled Antibody (Ab10-d2):

According to the manufacturer's information sheet, Ab10 binds to an epitope close to the binding site of EGFR since this antibody disrupts the binding of EGF to EGFR. The addition of excess EGF should also disrupt the binding of the antibody, thereby making it possible to estimate the nonspecific binding of this antibody by adding an excess of EGF.

Fluorescence measurements were carried out in fluorescence mode by exciting the acceptor at 640 nm and measuring its emission (“prompt fluorescence”) at 680 nm (given the bandwidth of the filters, one is entitled to put the measurements carried out with a “665 nm” and “680 nm” filter in the same category, in both cases, as a measurement corresponding to the “acceptor” channel).

| | |
|---|---|
| No. of flashes per well | 100 |
| Optical module | FI 640 680 |
| Excitation (nm) | 640 |
| Emission (nm) | 680 |
| Gain | 2459 |

The E680c measurements after subtraction of the buffer blank measured at 680 nm made it possible to obtain the concentrations, in nM, of antibody bound to the cells using a calibration range of Ab10-d2 (1 nM to 0.031 nM).

TABLE 8

| | | | |
|---|---|---|---|
| 9 | 10 | 11 | 12 |

| | | | | |
|---|---|---|---|---|
| 5 | 6 | 7 | 8 | +EGF (100 nM final |

| | | | |
|---|---|---|---|
| 3 | 4 | no EGF added | concentration) |

| | | | | | | | | |
|---|---|---|---|---|---|---|---|---|
| E680c | 102 800 | 105 900 | 71 500 | 91 500 | 53 800 | 65 350 | 54 750 | 62 800 |

| | | |
|---|---|---|
| Mean | 92 950 | 59 180 |
| E680c | | |
| Ab10-d2 | 6.74 | 4.29 |
| (nM) | | |

The signal/noise ratio when a single ligand of the receptor of interest is used (Ab10-d2) is therefore 1.58, which is relatively low.

### Measurement of the Binding of the Fluorescent Probes by TR-FRET:

In a similar manner, the signals at 620 nm and at 665 nm were measured in time-resolved mode in the wells containing dilutions of EGF-Lumi4 Tb and of Ab10-d2 (1 nM to 0.031 nM).

TABLE 9

| | | | | | | |
|---|---|---|---|---|---|---|
| E620c | 634 | 930 | 1 490 | 3 050 | 5 500 | 10 850 |
| E665c | 13 | 43 | 106 | 220 | 466 | 1 210 |

The straight line of correlation between E665c and E620c makes it possible to obtain the contribution of the emission of the terbium in the 665 nm channel: E665=0.102×E620, which makes it possible to obtain the B665 values corresponding to the contribution of the emission of the terbium.

The results of the time-resolved measurements at 665 nm (parameters above) have been grouped together in the following table, in which Δ665=E665c−B665.

TABLE 10

| | | | | | +EGF (100 nM final | | | |
|---|---|---|---|---|---|---|---|---|
| | No EGF added | | | | concentration) | | | |
| | Well | | | | | | | |
| | 5 | 6 | 7 | 8 | 9 | 10 | 11 | 12 |
| E665c | 2 060 | 2 390 | 1 600 | 1 860 | 390 | 480 | 290 | 320 |
| B665 | 395 | 480 | 345 | 335 | 120 | 137 | 79 | 93 |
| Δ665 | 1 660 | 1 910 | 1 260 | 1 520 | 270 | 340 | 215 | 223 |

| | | |
|---|---|---|
| Mean Δ665 | 1 588 | 262 |

In this case, the signal obtained at 665 nm represents the signal emitted by the acceptor (Ab10-d2) excited by FRET with the donor (EGF-Lumi4 Tb).

The signal/noise ratio when the method according to the invention is carried out, i.e. using two ligands labeled with FRET partners, is 6.0, which is higher than what is observed with the labeled ligand alone or the labeled antibody alone.

In order to take into account the variability in the number of cells in the wells, a normalization using the signal of Hoechst 33342 was carried out by dividing the Δ665 signal by the fluorescence value measured at 460 nm in “conventional” fluorescence mode for the Hoechst/DNA complex in each well (and by multiplying by 100 000 so as to obtain whole numbers).

TABLE 11

| | | | | | +EGF (100 nM final | | | |
|---|---|---|---|---|---|---|---|---|
| | No EGF added | | | | concentration) | | | |
| | Well | | | | | | | |
| | 5 | 6 | 7 | 8 | 9 | 10 | 11 | 12 |
| Δ665 | 1 660 | 1 910 | 1 260 | 1 520 | 270 | 340 | 215 | 223 |
| H460c | 67 380 | 99 330 | 79 590 | 106 300 | 82 990 | 109 800 | 74 400 | 88 000 |
| Δ665N | 2463 | 1923 | 1583 | 1430 | 325 | 309 | 289 | 253 |

| | | |
|---|---|---|
| Mean | 1 849 | 294 |
| Δ665N | | |

The signal/noise ratio is then 6.29, therefore higher than what is observed with the labeled ligand alone or the labeled antibody alone.

### Example 3

Cells of the A431 line (ATCC/CRL-1555 expressing approximately 2×10⁶ EGFR receptors per cell (Shinobu, 1984 Molecular and Cellular Endocrinology 37, 205)) were dispensed into the wells of a microtitration plate (flat-bottomed 96-well black plate) at a density of 25 000 cells per well and incubated over night at 37° C. in DMEM medium so as to obtain a layer of adherent cells.

Washing was carried out with 2×100 μl of PBS and then 50 μl of Krebs buffer were added per well.

[Krebs/HEPES buffer mmol/l: NaCl99; KCl4.69; CaCl₂ 1.87; MgSO₄ 1.2; K₂HPO₄ 1.03; NaHCO₃ 25; Na-HEPES 20+glucose 11.1 mM+0.1% BSA, pH 7.4].

No other reagent was added to wells B1 to B4 (buffer blank). A mixture of Ab10-d2 antibody (anti-EGFR Ab10 Thermo Fisher Scientific labeled with d2-NHS, Cisbio Bioassays) and of Cetuximab-Lumi4 Tb (anti-EGFR) was added to wells B5 to B8 and the volume was made up to 100 μl with Krebs buffer so as to obtain a final concentration in the wells of 5 nM of Ab10-d2 and 5 nM of Cetuximab-Lumi4 Tb.

In wells of the same microtitration plate, a standard range was formed by dilution, in Krebs buffer, of a stock solution containing EGF-Lumi4 Tb and Ab10-d2 (cascade ½ dilution so as to obtain final Ab concentrations of 1 nM to 0.031 nM). A time-resolved mode reading was carried out on a Pherastar FS device (BMG) using the following parameters:

| | |
|---|---|
| Number of flashes per well | 300 |
| Optical module | HTRF |
| Excitation (nm) | 337 |
| Emission A (nm) | 665 |
| Emission B (nm) | 620 |
| Start of integration [μs] | 60 |
| Integration time [μs] | 400 |
| Focusing height [mm] | 3.9 |
| Multiplier ratio | 10 000 |
| Light source | flash lamp |

In order to evaluate the nonspecific binding of the antibodies to the surface of the cells, CHO (Chinese Hamster Ovary) cells were incubated with the same solution of labeled antibodies, the wells were washed and the fluourecence was measured under the same conditions.

After washing, the mean signal emitted by Cetuximab-Lumi4 Tb alone is 24 033 AFU (arbitrary fluorescence units) for the A431 cells and 1170 AFU for the CHO cells, i.e. a signal/noise ratio of 21.

When the method according to the invention is carried out and the FRET signal is measured, a much better signal/noise ratio, of approximately 520, is obtained.

### Example 4

In this example, the method according to the invention was used to quantify the expression of the EGFR and HER2 proteins in samples of tumors from patients, in particular mammary tumors.

The method was carried out in a manner similar to example 1: the Lumi4®Tb and d2 fluorophores (Cisbio bioassays) were used as respectively donor and acceptor FRET partners. For measuring the expression of EGFR, the cetuximab (Merck KGaA) and Ab-10 (Thermo Scientific) antibodies, which both recognize distinct epitopes of EGFR, were used and labeled, respectively, with the Lumi4®Tb and d2 fluorophores. For quantifying HER2, the trastuzumab antibody (Roche Pharma AG) and the FRPS antibody (described by IM Harwerth et al. (1992) J. Biol. Chem. 267: 15160-15167), which are both specific for different epitopes of HER2, were also conjugated with Lumi4®Tb and d2, respectively.

For each assay, 50 nm tumor cryosections were incubated over night in 180 μl of TR-FRET buffer (1×PBS/10% BSA) containing 50 nM of each of the two antibodies. DNA staining was then carried out by adding 20 μl of a solution of Hoechst 33342 (Invitrogen) at 0.1 mg/ml and incubating at ambient temperature for 10 min. After washing and centrifugation, the samples were resuspended in the TR-FRET buffer, subjected to sonication and transferred into a microplate.

The Lumi4®Tb and d2 fluorescence signals were measured respectively at 620 and 665 nm in time-resolved mode (delay 60 μs; window 400 μs) after excitation at 337 nm using a Pherastar® fluorimeter (BMG Labtech). The Hoechst 33342 signal was measured in fluorescence mode at 460 nm. These signals were corrected with respect to the background noise according to the formula:

F_{corrected}=Fₛₐₘₚₗₑ−F_{background noise},

in which the F_{background noise} values were obtained by measuring the fluorescence of the TR-FRET buffer alone.

Furthermore, for each assay, the fluorescence signals measured on solutions obtained by cascade ½ dilution (so as to obtain a concentration range) from a stock solution containing a mixture of 50 nM of antibody—Lumi4® Tb and 50 nM of antibody—d2 were measured simultaneously with the samples, and a relationship was established between the signal obtained at 665 nm and that measured at 620 nm for each antibody concentration. The resulting curve was used to calculate the contribution of the Lumi4®-Tb fluorescence at 665 nm (F_{665Tb}) on the basis of the signal emitted by the samples at 620 nm. The TR-FRET signal was expressed in the following way:

ΔF₆₆₅=F₆₆₅ₛₐₘₚₗₑ−F_{665Tb}

The signal of the DNA-Hoechst 33342 complex at 460 nm (F₄₆₀) was used to normalize the TR-FRET signal so as to take into account the variability of the amount of biological material present in each measuring medium, with normalizing being at an average value of 100 000 fluorescence units (FU):

TR-FRET_{normalized}=(ΔF₆₆₅×100 000)/(F₄₆₀).

The normalized TR-FRET signal was expressed in FU.

In order to convert the normalized TR-FRET signal into number of receptors per cell, the number of receptors per cell was first of all evaluated on NIH/3T3 EGFR, NIH/3T3 HER2, NIH/3T3 EGFR/HER2 and SKOV-3 cell lines. For this, cells in culture were analyzed by FACS using an indirect quantitative immunofluorescence assay (QIFI kit, Dako) as described previously (Gaborit et al. 2011 J Biol Chem., 286(13):11337-45). In parallel, the expression of EGFR and HER2 was measured in samples of mouse xenografts derived from the corresponding tumor cells, using the TR-FRET assays. Thus, on the basis of the hypothesis according to which EGFR and HER2 are expressed at comparable levels in cell cultures and in xenografts of tumors derived from these cells, the values obtained in the xenografts were used as standards for converting the TR-FRET signal into number of receptors per cell.

### Results: Expression of EGFR and HER2

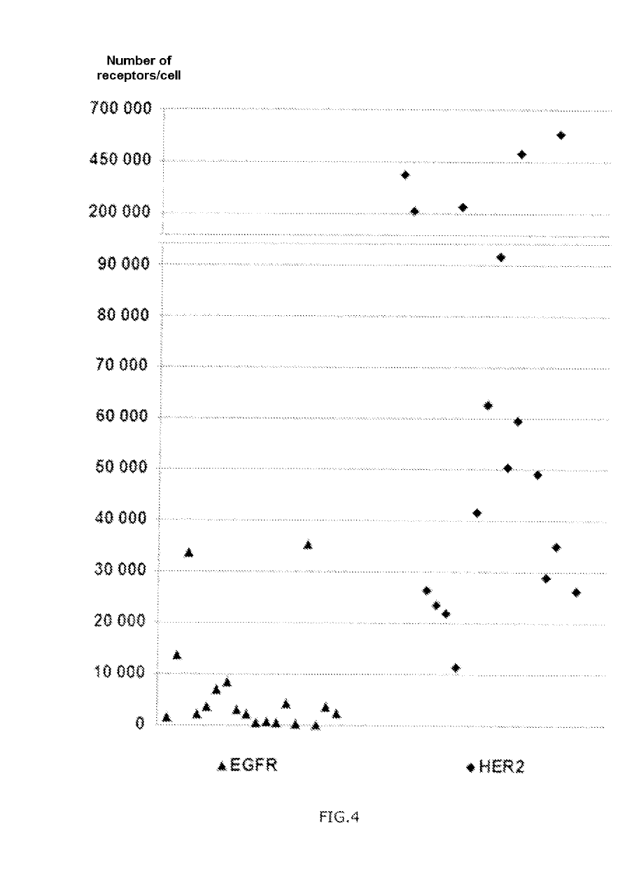

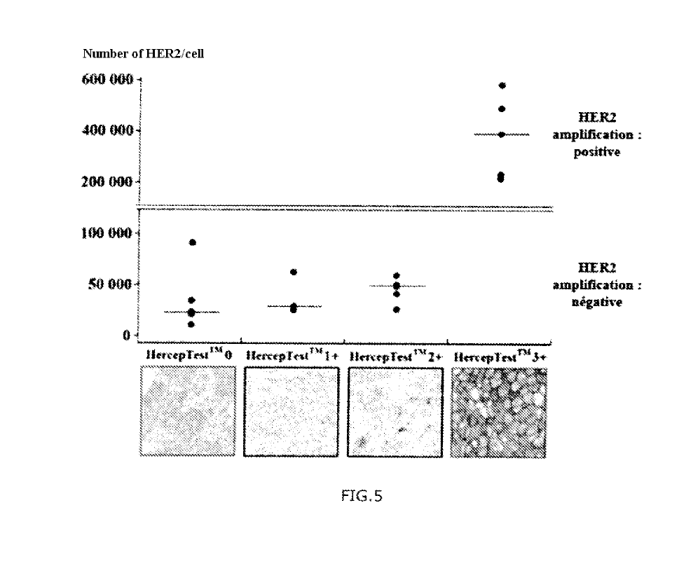

The results obtained with the 18 samples of mammary tumors are given in FIG. 4. The median levels of expression observed are 2800 EGFR/cell (range from 220 to 35 500) and 49 800 HER2/cell (range from 11 500 to 584 000). Five of the 18 tumors (i.e. 27.8%) express very high levels of HER2 (216 800, 234 300, 391 000, 491 500 and 584 000 HER2/cell). On average, the HER2 expression levels were 66 times higher than those of EGFR. The reproducibility of the results was verified by reproducing the experiments three times for each sample. The average coefficients of variation (CV) were 22% for EGFR and 19% for the HER2 quantification analysis. This variability takes into account the biological variability, since different cryosections were used for each experiment.

In order to confirm the EGFR quantification, the total RNA of each tumor was extracted for RT-qPCR analysis. A positive linear correlation (Rho=0.84, P<0.001) was observed between the EGFR protein expression levels determined by TR-FRET and the EGFR mRNA levels measured by RT-qPCR.

In order to validate the results of the HER2 quantification analysis using the method according to the invention, the expression of HER2 was evaluated using the HercepTest™ and also by measuring the amplification of the gene encoding HER2 by means of FISH and quantitative PCR analyses. The results of this analysis, given in FIG. 5, show no overlap in the expression levels determined by TR-FRET between the HER2-Herceptest™ positive tumors and the HER2-Herceptest™ negative tumors. Only the five breast tumors with >150 000 HER2/cell had a HercepTest™ score of 3+ and were positive for the HER2 gene amplification tests. This indicates that the method according to the invention makes it possible to detect the overexpression of HER2 with a 100% specificity and sensitivity in tumor samples, which makes it particularly invaluable for evaluating the susceptibility of patients to respond to certain anticancer treatments targeting HER2 (such as treatment with trastuzumab, Herceptin™)

For the first time, a reliable method for quantifying HER2 which can be carried out, for example, in hospital, makes it possible to evaluate the number of HER2 proteins per cell of patient samples. It does not have the drawbacks of immunohistochemical staining and of the FISH technique.

### Example 5

The expression of HER2 was quantified by the method described in example 4 on frozen samples of tumors from 100 patients suffering from breast cancer. The measurement of normalized fluorescence signals allowed a quantitative measurement of the expression of HER2 receptors. The disease-free survival (DFS) and the overall survival (OS) were evaluated for each patient.

### Result:

Among the 100 patients, 82 were IHC-HER2 negative (HercepTest™ negative), including 60 subjects who were ER (estrogen receptor) positive and treated with hormonal therapy. Using Cox proportional risk analyses, it was shown that, in the subjects who were IHC-HER2 negative and ER positive, the presence of HER2 was significantly associated both with a reduced DFS (p=0.0005) and a reduced OS (p=0.003).

The quantitative measurement of the expression of HER2 using the method of the invention can make it possible to predict the outcome of the disease in subjects suffering from breast cancer who are IHC-HER2 negative and ER positive. This biomarker may be useful for identifying patients whose hormonal treatment is not sufficiently effective and who might benefit from an adjuvant treatment by anti-HER therapy of Herceptin™ type. One of the major contributions of the invention is to be able to improve the selection of patients who may be able to respond to this type of treatment.

1. A method for quantifying a protein of interest expressed in a tissue sample, comprising the following steps:
(i) bringing a tissue sample expressing said protein into contact with a first and a second ligand, each of these ligands being capable of binding specifically to a domain of said protein, and these ligands being respectively labeled with a donor compound and an acceptor compound, both forming a pair of FRET partners;
(ii) washing the tissue sample;
(iii) measuring the FRET signal emitted by the measuring medium.

2. The method as claimed in claim 1, which further comprises, prior to the washing step, a step of incubating the tissue sample with an agent for fluorescently labeling of DNA, wherein the FRET signal is normalized with respect to the signal corresponding to the luminescence of this labeling agent.

3. The method as claimed in claim 1, wherein said first and second ligands are antibodies.

4. The method as claimed in claim 1, wherein the first ligand is a known ligand of the protein of interest and is not an antibody, and wherein the second ligand is an antibody.

5. The method as claimed in claim 1, wherein the protein of interest is a membrane protein.

6. The method as claimed in claim 1, wherein the protein of interest is selected from the group consisting of a G-protein-coupled membrane receptor and a receptor tyrosine kinase.

7. The method as claimed in claim 1, wherein the protein of interest is selected from the group consisting of EGFR, HER2, HER3 and HER4.

8. The method as claimed in claim 1, wherein the donor compound is a rare earth chelate or cryptate.

9. The method as claimed in claim 8, wherein the donor compound is a europium chelate or cryptate or a terbium chelate or cryptate.

10. The method as claimed in claim 1, wherein the acceptor compound is selected from the group consisting of allophycocyanins, rhodamines, cyanines, squaraines, coumarins, proflavins, acridines, fluoresceins, boron-dipyrromethene derivatives, fluorophores known under the name “Atto”, fluorophores known under the name “DY”, compounds known under the name “Alexa” and nitrobenzoxadiazole.

11. The method as claimed in claim 1, which is carried out on a solid tissue sample.

12. The method as claimed in claim 1, which is carried out with a solid tissue sample and wherein said first and second ligands are introduced into the measuring medium at a final concentration greater than 10 nM.

13. The method as claimed in claim 1, which is carried out with a solid tissue sample wherein said first and second ligands are introduced into the measuring medium at a final concentration of between 20 and 80 nM.

14. The method as claimed in claim 1, which is carried out on a solid tissue sample and which further comprises a step of homogenizing this sample in the form of a cell lysate.

15. The method as claimed in claim 14, wherein the step of homogenizing the solid tissue sample is carried out after the introduction of the first and second ligands, and before the measurement of the FRET signal.

16. The method as claimed in claim 1, which is carried out with a tumor tissue sample.

17. The method as claimed in claim 1, wherein, when the first or the second ligand is an antibody, the epitope of said antibody is located on a domain of the protein of interest that is exposed to the extracellular medium.

18. The method as claimed in claim 1, wherein the protein of interest is the HER2 protein, and wherein the first and second ligands are antibodies specific for this protein.

19. A kit of reagents for carrying out the method as claimed in claim 1, which contains a first and a second ligand, each of these ligands being capable of binding specifically to a domain of a membrane protein of interest, wherein these ligands are respectively labeled with a donor compound and an acceptor compound, which donor and acceptor compounds form a pair of FRET partners.

20. The kit of reagents as claimed in claim 19, wherein the ligands are antibodies specific for the EGFR receptor.

21. The kit of reagents as claimed in claim 19, wherein the ligands are antibodies specific for the HER2 protein.

22. An ex vivo method for determining whether a patient is eligible for a therapeutic treatment with an antibody which binds to the HER2 protein, which comprises carrying out a method of quantification as claimed in claim 1 in which the protein of interest is the HER2 protein.

23. The method as claimed in claim 22, which is carried out with a tissue sample from a patient for whom the results of immunohistochemical analysis of the expression of the HER2 protein are negative.
